(19) United States
(12) Patent Application Publication
(10) Pub. No.: US 2024/0350596 A1
(43) Pub. Date: Oct. 24, 2024

(54) DOWNREGULATION OF CIRCULATING GHRELIN AND THERAPEUTIC APPLICATIONS THEREOF

(71) Applicant: University of Kentucky Research Foundation, Lexington, KY (US)

(72) Inventors: Chang-Guo Zhan, Lexington, KY (US); Fang Zheng, Lexington, KY (US)

(21) Appl. No.: 18/685,208

(22) PCT Filed: Aug. 24, 2022
(86) PCT No.: PCT/US22/75391

§ 371 Date: Feb. 20, 2024

Related U.S. Application Data
(60) Provisional application No. 63/236,532, filed on Aug. 24, 2021.

(51) Int. Cl. A61K 38/46; A61P 25/30; A61P 25/34; C12N 9/18

(52) U.S. Cl. CPC A61K 38/465; A61P 25/30; A61P 25/34; C12N 9/18; C12Y 301/01008; C07K 2319/30

(57) ABSTRACT

Compositions and methods for converting ghrelin to desacyl-ghrelin include a butyrylcholinesterase (BChE) polypeptide variant. The presently-disclosed subject matter includes compositions and methods for use in directly downregulating ghrelin. In particular, certain embodiments relate to compositions and methods for hydrolyzing/deacylating ghrelin to desacyl-ghrelin and applications thereof in the treatment of disorders involving ghrelin.

### RELATED APPLICATIONS

This application claims priority from U.S. Provisional Application Ser. No. 63/236,532 filed Aug. 24, 2021, the entire disclosure of which is incorporated herein by this reference.

### GOVERNMENT INTEREST

This invention was made with government support under grant number U18 DA052319 awarded by the National Institutes of Health. The government has certain rights in the invention.

### TECHNICAL FIELD

The presently-disclosed subject matter generally relates to compositions and methods for use in downregulating ghrelin. In particular, certain embodiments of the presently-disclosed subject matter relate to compositions and methods for hydrolyzing/deacylating ghrelin to desacyl-ghrelin and application thereof in the treatment of disorders involving ghrelin. Certain embodiments of the presently-disclosed subject matter relate to compositions and methods for treating substance use disorders, including polysubstance use disorders.

### INTRODUCTION

Ghrelin is a 28 amino-acid peptide (SEQ ID NO: 1), which is acylated (n-octanoylation) at Ser3 side chain by ghrelin O-acyl transferase (GOAT).¹⁻² Without acylation, the peptide is known as desacyl-ghrelin. Desacyl-ghrelin does not bind with or activate ghrelin receptor, which is also known as growth hormone (GH) secretagogue receptor (GHSR).³

While the GOAT converts desacyl-ghrelin to ghrelin, ghrelin is also hydrolyzed/deacylated to desacyl-ghrelin by circulating esterases, particularly wild type butyrylcholinesterase (BChE) (SEQ ID NO: 2).⁴⁻⁵ Plasma BChE is the main enzyme responsible for converting ghrelin to desacyl-ghrelin, although other enzymes including platelet-activating factor acetylhydrolase may also be able to convert ghrelin to desacyl-ghrelin.⁶⁻⁷ Due to the co-existence of enzymes GOAT and BChE etc., ghrelin and desacyl-ghrelin co-exist in the body, but only ghrelin is able to bind and activate GHSR.

Based on the data reported in the literature, ghrelin levels rise steeply with fasting or before a meal, and decrease after a meal.⁸ According to reported ghrelin level data, most plasma ghrelin molecules exist in the desacyl-ghrelin form, with only ˜10%⁹ to ˜30%¹⁰ of circulating ghrelin being acylated. For convenience, throughout this document, the term “ghrelin” refers to acylated ghrelin, unless explicitly stated otherwise.

Gastric endocrine cells synthesize and secrete most of the body's ghrelin into the bloodstream.^{1,11} Ghrelin can cross the blood-brain barrier (BBB) to activate its receptor GHSR in the brain.¹

It is known that ghrelin is related to a variety of disorders. With regard to obesity, for example, ghrelin is also known as a gastric peptide hormone or the “hunger hormone”.¹ So far, ghrelin is the only known hormone stimulating hunger and food intake, letting one know when to eat. Therefore, ghrelin is emerging as a novel, potentially attractive anti-obesity drug target.¹² Obesity-related drug discovery efforts centralized on ghrelin aim to reduce a subject's appetite by different approaches, including regulation of ghrelin release, ghrelin receptor antagonism, and reducing active ghrelin production by inhibition of GOAT.¹³⁻¹⁷

With regard to other conditions, it has been known that plasma ghrelin is elevated in hyperphagia and Prader-Willi syndrome (PWS).¹⁸ Therefore, high circulating ghrelin was proposed as the potential cause of hyperphagia and PWS.¹⁸ Concerning hyperglycemia, it was reported that in individuals with obesity, ghrelin reduces insulin sensitivity and, thus, contributes to hyperglycemia and worsened glucose intolerance.¹⁹

For another example, sleep disorder is also related to elevated ghrelin.²⁰ It has been demonstrated that downregulation of ghrelin is a promising strategy for treatment of emotional disorder.²¹ For another example, it is known that ghrelin inhibition is considered as a promising strategy for treatment of type 2 diabetes (T2D).²² Ghrelin also plays an important role in several key processes of cancer progression, including cell proliferation, migration and invasion.²³ There is a correlation of ghrelin with poor clinical outcome (poor survival outcome).²³

In addition, ghrelin plays a role in substance use disorders.²⁷⁻³² It is known that about 10 percent of American adults have struggled with substance use disorders (SUDs).²⁴ In some SUDs, such as methamphetamine (METH) and cocaine use disorders, there are no FDA (Food and Drug Administration)-approved medications. For other substances, such as alcohol, nicotine, and opioids (e.g. heroin, morphine, oxycodone, hydrocodone, fentanyl and its derivatives), there are FDA-approved medications available, but relapse rates are high, necessitating new treatment options.²⁵⁻²⁶

Current therapeutic development efforts underway in academia and industry have been focused mainly on small-molecule compounds that can bind with specific receptors/transporters (targets) in the brain to attenuate the physiological, subjective, and/or reinforcing effects of the drugs, although there are also efforts to develop biologics including drug-binding monoclonal antibodies or vaccines that produce the desirable antibodies that can bind tightly to the drugs so as to prevent crossing BBB.

One of the recognized promising targets is GHSR,²⁷⁻³² as reported studies have consistently demonstrated that ghrelin activating GHSR is necessary for substance reward. It has been demonstrated that administration of a selective GHSR antagonist (JMV2959)³³ significantly and dose-dependently attenuated morphine-induced conditioned place preference (CPP) and dopamine sensitization, and also attenuated METH self-administration, tendency to relapse, and METH-induced CPP.²⁷⁻³² It has also been demonstrated in a most recently reported study.³⁴ that pretreatment with JMV2959 significantly and dose-dependently reduced the manifestation of fentanyl-CPP and reduced the fentanyl-seeking/relapse-like behavior tested in rats on the 12th day of the forced abstinence period.

In fact, GHSR antagonism is one of the NIDA's 10 Most-Wanted targets³⁵ for opioid use disorder medication development. On the other hand, GHSR possesses a naturally high constitutive activity (which is the GHSR activity in the absence of ghrelin ligand) representing 50% of its maximal activity.³⁶ Due to the ghrelin receptor's diverse regulatory roles associated with the constitutive activity,³⁷ GHSR antagonism could also result in unwanted adverse effects.³⁸

Accordingly, ghrelin is an interesting therapeutic target related to a variety of disorders, such as obesity, hyperphagia and Prader-Willi syndrome, hyperglycemia, sleep disorder, emotional disorder, type 2 diabetes (T2D), cancer, and substance use disorders. Approaches-to-date have been directed to: regulation of ghrelin release, ghrelin receptor/GHSR antagonism, and reducing active ghrelin production by inhibition of GOAT. However, these approaches not seek to directly inactivate ghrelin, and have the potential for undesirable adverse effects.

Accordingly, there is a need in the art for compositions and methods for use in downregulating ghrelin, which can be used in the treatment of ghrelin-related disorders.

### SUMMARY

The presently-disclosed subject matter meets some or all of the above-identified needs, as will become evident to those of ordinary skill in the art after a study of information provided in this document.

The presently-disclosed subject matter includes compositions and methods for use in directly downregulating ghrelin. In particular, certain embodiments of the presently-disclosed subject matter relate to compositions and methods for hydrolyzing/deacylating ghrelin to desacyl-ghrelin and applications thereof in the treatment of disorders involving ghrelin.

Previously described approaches to targeting ghrelin have involved regulation of ghrelin release, ghrelin receptor/GHSR antagonism, and reducing active ghrelin production by inhibition of ghrelin O-acyl transferase (GOAT). However, these approaches not seek to directly inactivate ghrelin, and have the potential for undesirable adverse effects.

In this regard, in a ghrelin-related disorder, inhibition of GOAT would decrease the production of (active) ghrelin, but cannot inactivate ghrelin that has already been produced in the body. The presently-disclosed subject matter addresses this issue, and others identified herein, by providing compositions and methods for directly downregulating ghrelin. Directly-inactivating ghrelin using the presently-disclosed compositions and methods involves converting ghrelin into a peptide form (desacyl-ghrelin), which is devoid of the active properties of ghrelin. In this regard, a ghrelin hydrolase can directly inactivate ghrelin, including ghrelin that has already been produced by the body, by converting it into desacyl-ghrelin, thereby affecting the ghrelin-related disorder.

The presently-disclosed subject matter is based, at least in part, on the present inventors' discovery that certain polypeptide molecules, as disclosed herein, have beneficial ghrelin hydrolase activities. The presently-disclosed subject matter includes compositions and methods that make use of these polypeptide molecules, as disclosed herein.

The presently-disclosed subject matter includes compositions and methods of inactivating ghrelin using butyrylcholinesterase (BChE) or a BChE polypeptide variant as disclosed herein. The compositions and methods as disclosed herein can be used for the treatment of conditions, such as obesity, hyperphagia and Prader-Willi syndrome, hyperglycemia, sleep disorder, emotional disorder, type 2 diabetes (T2D), cancer, and substance use disorders. In some embodiments, the compositions and methods as disclosed herein can be used for the treatment of polysubstance use disorders.

The presently-disclosed subject matter includes use of BChE or a BChE polypeptide variant as disclosed herein in a medicament for converting ghrelin to desacyl-ghrelin. The presently-disclosed subject matter includes use of BChE or a BChE polypeptide variant as disclosed herein in a medicament for treating obesity, hyperphagia and Prader-Willi syndrome, hyperglycemia, sleep disorder, emotional disorder, type 2 diabetes (T2D), cancer, and substance use disorders. The presently-disclosed subject matter includes use of BChE or a BChE polypeptide variant as disclosed herein in a medicament for treating polysubstance use disorders.

The presently-disclosed subject matter further includes polypeptide molecules and nucleotide molecules encoding polypeptide molecules for BChE polypeptide variants that can be used as a ghrelin hydrolase.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1A-1C. Effects of enzyme (ghrelin deacylase BChE-M02) on plasma ghrelin level (FIG. 1A) and methamphetamine (METH)-induced hyperactivity (FIG. 1B) in mice (n=8/group). The enzyme at a dose of 26 mg/kg (or saline) was injected intravenously (IV) three hours before IP injection of 0.5 mg/kg METH (or saline). Blood samples were collected at the end of the locomotor activity testing (five hours after the enzyme or saline injection). METH (0.5 mg/kg, IP)-induced conditioned placed preference (CPP) expressed as % in control and treatment groups (FIG. 1C).

FIG. 2A-2C. Effects of ghrelin deacylase (BChE-M02Fc) on morphine-(FIG. 2A) and fentanyl-(FIG. 2B) induced hyperactivity in mice (n=8/group). The enzyme at a dose of 15 mg/kg (or saline) was injected (IV) three hours before IP injection of 10 mg/kg morphine (or saline) or 0.1 mg/kg fentanyl (or saline). (C) Fentanyl (0.1 mg/kg, IP)- and morphine (10 mg/kg, IP)-induced CPP in mice (n=8/group) expressed as % in control and treatment groups (FIG. 2C).

FIG. 3. Effect of recombinant wild-type BChE fused with Fc of human IgG1 (denoted as wtBChE-Fc) on METH-induced hyperactivity in mice (n=8 per group). Mice were given wtBChE-Fc (26 mg/kg, IV) 2 h before the METH administration.

FIG. 4. Insulin level in morphine-challenged mice (n=8/group). A single dose of enzyme (BChE-M02Fc. 15 mg/kg, IV) was injected at 3 h before IP injection of 10 mg/kg morphine on Day 0, followed by daily 10 mg/kg morphine (IP) challenge on Days 1 to 5. Blood samples were collected at 2 h after the morphine administration. Fasting time 10:00 am-12:00 pm.

FIG. 5. Glucose level in morphine-challenged mice (n=8/group). A single dose of enzyme (15 mg/kg, IV) was injected at 3 h before IP injection of 10 mg/kg morphine on Day 0, followed by daily 10 mg/kg morphine (IP) challenge on Days 1 to 5. Blood samples were collected at 2 h after the morphine administration. Average baseline glucose level: 1.19 g/L. Fasting time: 10:00 am-12:00 pm.

### BRIEF DESCRIPTION OF THE SEQUENCE LISTING

SEQ ID NO: 1 is an amino acid sequence for wild type ghrelin.

SEQ ID NO: 2 is an amino acid sequence including the 574 amino acid sequence of wild type butyrylcholinesterase (BChE).

SEQ ID NO: 3 is an amino acid sequence for a truncated BChE polypeptide variant having the following amino acid substitution, as compared to residues 1-529 of SEQ ID NO: 2: F398I.

SEQ ID NO: 4 is an amino acid sequence for a truncated BChE polypeptide variant having the following amino acid substitutions, as compared to residues 1-529 of SEQ ID NO: 2: T284S/P285I/F398I.

SEQ ID NO: 5 is an amino acid sequence for a truncated BChE polypeptide variant having the following amino acid substitutions, as compared to residues 1-529 of SEQ ID NO: 2: T284S/P285I/V288I.

SEQ ID NO: 6 is an amino acid sequence for a truncated BChE polypeptide variant having the following amino acid substitutions, as compared to residues 1-529 of SEQ ID NO: 2: T284S/P285I/V288I/F398I.

SEQ ID NO: 7 is an amino acid sequence for a truncated BChE polypeptide variant having the following amino acid substitutions, as compared to residues 1-529 of SEQ ID NO: 2: A199S/S287G/A328W/Y332G.

SEQ ID NO: 8 is an amino acid sequence for a truncated BChE polypeptide variant having the following amino acid substitutions, as compared to residues 1-529 of SEQ ID NO: 2: A199S/F227A/P285Q/S287G/A328W/Y332G.

SEQ ID NO: 9 is an amino acid sequence for a truncated BChE polypeptide variant having the following amino acid substitutions, as compared to residues 1-529 of SEQ ID NO: 2: A199S/F227A/P285S/S287G/A328W/Y332G.

SEQ ID NO: 10 is an amino acid sequence for a truncated BChE polypeptide variant having the following amino acid substitutions, as compared to residues 1-529 of SEQ ID NO: 2: A199S/F227A/P285A/S287G/A328W/Y332G.

SEQ ID NO: 11 is an amino acid sequence for a BChE polypeptide variant known as CocH3, having the following amino acid substitutions, as compared to SEQ ID NO: 2: A199S/F227A/S287G/A328W/Y332G.

SEQ ID NO: 12 is an amino acid sequence of wild type Fc polypeptide.

SEQ ID NO: 13 is an amino acid sequence of an embodiment of a linker.

SEQ ID NO: 14 is an amino acid sequence of an embodiment of a linker.

SEQ ID NO: 15 is an amino acid sequence of an embodiment of a linker.

SEQ ID NO: 16 is an amino acid sequence of an embodiment of a linker.

SEQ ID NO: 17 is an amino acid sequence of an embodiment of a linker.

SEQ ID NO: 18 is an amino acid sequence of an embodiment of a linker.

This application contains a sequence listing submitted in accordance with 37 C.F.R. 1.821, named “Zhan UKRF 2584_ST25.txt”, having a size of 50 kilobytes, which is incorporated herein by this reference.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter is based, at least in part, on the present inventors' discovery that certain polypeptide molecules, as disclosed herein, have beneficial ghrelin hydrolase activities. The presently-disclosed subject matter includes compositions and methods that make use of these polypeptide molecules, as disclosed herein.

The presently-disclosed subject matter is based, at least in part, on the present inventors' discovery that certain polypeptide molecules, as disclosed herein, have beneficial ghrelin hydrolase activities. The presently-disclosed subject matter includes compositions and methods that make use of these polypeptide molecules, as disclosed herein.

The presently-disclosed subject matter includes compositions and methods of inactivating ghrelin using butyrylcholinesterase (BChE) or a BChE polypeptide variant as disclosed herein. The presently-disclosed subject matter includes use of BChE or a BChE polypeptide variant as disclosed herein in a medicament for converting ghrelin to desacyl-ghrelin. The presently-disclosed subject matter also includes compositions and methods, and use of BChE or a BChE polypeptide variant as disclosed herein in a medicament, for treating obesity, hyperphagia and Prader-Willi syndrome, hyperglycemia, sleep disorder, emotional disorder, type 2 diabetes (T2D), cancer, and substance use disorders. In some embodiments, the compositions as disclosed herein include a BChE or BChE polypeptide variant and a pharmaceutically-acceptable carrier.

The presently-disclosed subject matter is based, at least in part, on the present inventors' discovery that administration of an exogenous ghrelin deacylase (BChE polypeptide variants as disclosed herein) can serve as an effective therapeutic strategy to treat substance use disorders (SUDs). As disclosed herein, the administration of an exogenous ghrelin deacylase can significantly and effectively attenuate the physiological effects of substances of abuse (See Examples, locomotor activity data) and their reward effects (See Examples, CPP data). Since it has been demonstrated, as disclosed herein, that the ghrelin deacylase can effectively attenuate the reward and physiological effects of multiple types of substances of abuse, this unique therapeutic approach can be used for polysubstance use disorders.

In this regard, in some embodiments, the compositions and methods as disclosed herein can be used for the treatment of polysubstance use disorders. The presently-disclosed subject matter also includes use of BChE or a BChE polypeptide variant as disclosed herein in a medicament for treating polysubstance use disorders. In some embodiments, a subject is in need of treatment for polysubstance use disorder involving two or more substances selected from the group consisting of: methamphetamine, cocaine, alcohol, nicotine, and opioids.

In some embodiments of the compositions, methods, and uses of the presently-disclosed subject matter, the BChE or BChE polypeptide variant is administered to a subject in an effective amount to downregulate circulating ghrelin in the plasma of the subject.

The presently-disclosed subject matter further includes polypeptide molecules and nucleotide molecules encoding polypeptide molecules for BChE polypeptide variants that can be used as a ghrelin hydrolase. In some embodiments, the BChE polypeptide variant includes amino acid substitutions as set forth in Table A, relative to SEQ ID NO: 2.

TABLE A
Substitutions relative to SEQ ID NO: 2 for Exemplary BChE Polypeptide Variants

| | | | | | | | | | | | Amino Acids |
|---|---|---|---|---|---|---|---|---|---|---|---|
| 199 | 227 | 284 | 285 | 286 | 287 | 288 | 328 | 332 | 398 | 441 | Included |
| A199S | — | — | — | — | — | — | A328W | Y332G | — | — | 1-574 |
| A199S | — | — | — | — | — | — | A328W | Y332G | — | — | 68-442 |
| A199S | — | — | — | — | — | — | A328W | Y332G | — | — | 117-438 |
| A199S | F227A | — | — | — | — | — | A328W | Y332G | — | — | 1-574 |
| A199S | F227A | — | — | — | — | — | A328W | Y332G | — | — | 68-442 |
| A199S | F227A | — | — | — | — | — | A328W | Y332G | — | — | 117-438 |
| A199S | — | — | — | — | S287G | — | A328W | Y332G | — | — | 1-574 |
| A199S | — | — | — | — | S287G | — | A328W | Y332G | — | — | 68-442 |
| A199S | — | — | — | — | S287G | — | A328W | Y332G | — | — | 117-438 |
| A199S | F227A | — | — | — | S287G | — | A328W | Y332G | — | — | 1-574 |
| A199S | F227A | — | — | — | S287G | — | A328W | Y332G | — | — | 68-442 |
| A199S | F227A | — | — | — | S287G | — | A328W | Y332G | — | — | 117-438 |
| A199S | F227A | — | — | — | S287G | — | A328W | — | — | E441D | 1-574 |
| A199S | F227A | — | — | — | S287G | — | A328W | — | — | E441D | 68-442 |
| A199S | F227A | — | — | — | S287G | — | A328W | — | — | E441D | 117-438 |
| A199S | F227A | — | P285A | — | S287G | — | A328W | Y332G | — | — | 1-574 |
| A199S | F227A | — | P285S | — | S287G | — | A328W | Y332G | — | — | 1-574 |
| A199S | F227A | — | P285Q | — | S287G | — | A328W | Y332G | — | — | 1-574 |
| A199S | F227P | — | — | — | S287G | — | A328W | Y332G | — | — | 1-574 |
| A199S | F227A | — | P285G | — | S287G | — | A328W | Y332G | — | — | 1-574 |
| A199S | F227A | — | — | L286M | S287G | — | A328W | Y332G | — | — | 1-574 |
| A199S | — | — | P285Q | — | S287G | — | A328W | Y332G | — | — | 1-574 |
| A199S | — | — | P285I | — | S287G | — | A328W | Y332G | — | — | 1-574 |
| A199S | F227G | — | — | — | S287G | — | A328W | Y332G | — | — | 1-574 |
| A199S | — | — | P285S | — | S287G | — | A328W | Y332G | — | — | 1-574 |
| A199S | F227V | — | — | — | S287G | — | A328W | Y332G | — | — | 1-574 |
| A199S | — | — | P285G | — | S287G | — | A328W | Y332G | — | — | 1-574 |
| A199S | F227I | — | — | — | S287G | — | A328W | Y332G | — | — | 1-574 |
| A199S | F227L | — | — | — | S287G | — | A328W | Y332G | — | — | 1-574 |
| A199S | — | — | — | L286M | S287G | — | A328W | Y332G | — | — | 1-574 |
| A199S | F227A | — | P285K | — | S287G | — | A328W | Y332G | — | — | 1-574 |
| A199S | F227S | — | — | — | S287G | — | A328W | Y332G | — | — | 1-574 |
| A199S | F227T | — | — | — | S287G | — | A328W | Y332G | — | — | 1-574 |
| A199S | F227M | — | — | — | S287G | — | A328W | Y332G | — | — | 1-574 |
| A199S | F227C | — | — | — | S287G | — | A328W | Y332G | — | — | 1-574 |
| A199S | F227A | — | P285N | — | S287G | — | A328W | Y332G | — | — | 1-574 |
| A199S | F227P | — | P285A | — | S287G | — | A328W | Y332G | — | — | 1-574 |
| A199S | F227S | — | P285Q | — | S287G | — | A328W | Y332G | — | — | 1-574 |
| A199S | F227S | — | P285S | — | S287G | — | A328W | Y332G | — | — | 1-574 |
| A199S | F227S | — | P285G | — | S287G | — | A328W | Y332G | — | — | 1-574 |
| A199S | F227P | — | P285S | L286M | S287G | — | A328W | Y332G | — | — | 1-574 |
| A199S | F227A | — | P285S | — | S287G | — | A328W | — | — | E441D | 1-574 |
| A199S | F227A | — | P285A | — | S287G | — | A328W | — | — | E441D | 1-574 |
| A199S | F227P | — | — | L286M | S287G | — | A328W | Y332G | — | — | 1-574 |
| A199S | F227G | — | P285A | — | S287G | — | A328W | Y332G | — | — | 1-574 |
| A199S | F227G | — | P285G | — | S287G | — | A328W | Y332G | — | — | 1-574 |
| A199S | F227G | — | P285Q | — | S287G | — | A328W | Y332G | — | — | 1-574 |
| A199S | F227G | — | P285S | — | S287G | — | A328W | Y332G | — | — | 1-574 |
| A199S | F227A | — | P285E | — | S287G | — | A328W | Y332G | — | — | 1-574 |
| A199S | F227P | — | P285N | — | S287G | — | A328W | Y332G | — | — | 1-574 |
| A199S | F227S | — | P285A | — | S287G | — | A328W | Y332G | — | — | 1-574 |
| A199S | F227S | — | P285N | — | S287G | — | A328W | Y332G | — | — | 1-574 |
| A199S | F227S | — | — | L286M | S287G | — | A328W | Y332G | — | — | 1-574 |
| A199S | F227G | — | — | L286M | S287G | — | A328W | Y332G | — | — | 1-574 |
| A199S | F227A | — | — | — | S287G | — | A328W | Y332G | — | — | 1-529 |
| A199S | F227A | — | — | — | S287G | — | A328W | Y332G | — | — | 1-574 |
| A199S | F227A | — | — | — | S287G | — | A328W | Y332G | — | — | 1-530 |
| A199S | F227A | — | — | — | S287G | — | A328W | Y332G | — | — | 1-531 |
| A199S | F227A | — | — | — | S287G | — | A328W | Y332G | — | — | 1-532 |
| A199S | F227A | — | — | — | S287G | — | A328W | Y332G | — | — | 1-533 |
| A199S | F227A | — | — | — | S287G | — | A328W | Y332G | — | — | 1-534 |
| A199S | F227A | — | — | — | S287G | — | A328W | Y332G | — | — | 1-535 |
| A199S | F227A | — | — | — | S287G | — | A328W | Y332G | — | — | 1-536 |
| A199S | F227A | — | — | — | S287G | — | A328W | Y332G | — | — | 1-537 |
| A199S | F227A | — | — | — | S287G | — | A328W | Y332G | — | — | 1-538 |
| A199S | F227A | — | — | — | S287G | — | A328W | Y332G | — | — | 1-539 |
| A199S | F227A | — | — | — | S287G | — | A328W | Y332G | — | — | 1-540 |
| A199S | F227A | — | P285Q | — | S287G | — | A328W | Y332G | — | — | 1-529 |
| A199S | F227A | — | P285Q | — | S287G | — | A328W | Y332G | — | — | 1-536 |
| — | — | — | — | — | — | — | — | — | F398I | — | 1-529 |
| — | — | T284S | P285I | — | — | — | — | — | F398I | — | 1-529 |
| — | — | T284S | P285I | — | — | V288I | — | — | — | — | 1-529 |
| — | — | T284S | P285I | — | — | V288I | — | — | F398I | | 1-529 |
| A199S | — | — | — | — | S287G | — | A328W | Y332G | — | — | 1-529 |
| A199S | F227A | — | P285Q | — | S287G | — | A328W | Y332G | — | — | 1-529 |
| A199S | F227A | — | P285S | — | S287G | — | A328W | Y332G | — | — | 1-529 |
| A199S | F227A | — | P285A | — | S287G | — | A328W | Y332G | — | — | 1-529 |

In some embodiments of the presently-disclosed subject matter, the BChE polypeptide variant comprises an amino acid sequence having 1, 2, 3, 4, 5, 6, 7, 8, 9, 10, or 11 mutations relative to the amino acid sequence of SEQ ID NO: 2, said mutations occurring at residues selected from the group consisting of 199, 227, 284, 285, 286, 287, 288, 328, 332, 398, and 441, and wherein the amino acid sequence of the BChE polypeptide variant is optionally truncated relative to the amino acid sequence of SEQ ID NO: 2, such that one or more residues from 1 to 67 and/or one or more residues from 443 to 574 is truncated.

In some embodiments of the presently-disclosed subject matter, the BChE polypeptide variant comprises an amino acid sequence having 1, 2, 3, 4, 5, or 6 mutations relative to the amino acid sequence of SEQ ID NO: 2, said mutations selected from the group consisting of A199S, F227A, T284S, P285I or P285A or P285Q or P285S, S287G, V288I, A328W, Y332G, and F398I, and wherein the amino acid sequence of the BChE polypeptide variant is optionally truncated relative to the amino acid sequence of SEQ ID NO: 2, such that one or more residues from 1 to 67 and/or one or more residues from 443 to 574 is truncated.

In some embodiments of the presently-disclosed subject matter, the BChE polypeptide variant comprises an amino acid sequence having 1, 2, 3, or 4 mutations relative to the amino acid sequence of SEQ ID NO: 2, said mutations selected from the group consisting of T284S, P285I. V288I, F398I; and wherein the amino acid sequence of the BChE polypeptide variant is optionally truncated relative to the amino acid sequence of SEQ ID NO: 2, such that one or more residues from 1 to 67 and/or one or more residues from 443 to 574 is truncated.

In some embodiments of the presently-disclosed subject matter, the BChE polypeptide variant comprises an amino acid sequence having 1, 2, 3, 4, 5, or 6 mutations relative to the amino acid sequence of SEQ ID NO: 2, said mutations selected from the group consisting of A199S, F227A, P285A or P285Q or P285S, S287G, A328W, and Y332G; and wherein the amino acid sequence of the BChE polypeptide variant is optionally truncated relative to the amino acid sequence of SEQ ID NO: 2, such that one or more residues from 1 to 67 and/or one or more residues from 443 to 574 is truncated.

In some embodiments of the presently-disclosed subject matter, the BChE polypeptide variant comprises an amino acid sequence wherein residues 530 to 574 are truncated, such that the amino acid sequence has 529 amino acids.

In some embodiments of the presently-disclosed subject matter, the BChE polypeptide variant comprises the amino acid sequence of any one of SEQ ID NOS: 3-10.

In some embodiments of the presently-disclosed subject matter, a Fc polypeptide is joined to an N- or C-terminal end of the BChE polypeptide variant. In some embodiments, the Fc polypeptide has the sequence of SEQ ID NO: 12, or a fragment thereof, wherein the Fc polypeptide or fragment thereof includes 0 to 8 amino acid substitutions at 0 to 8 of residues selected from 1, 6, 12, 15, 24, 38, 40, 42, 58, 69, 80, 98, 101, 142, and 144.

In some embodiments of the present invention, the Fc polypeptide has the sequence of SEQ ID NO: 12, or a fragment thereof, wherein the Fc polypeptide or fragment thereof includes 3 to 8 amino acid substitutions at 3 to 8 of residues selected from 1, 6, 12, 15, 24, 38, 40, 42, 58, 69, 80, 98, 101, 142, and 144. In further embodiments of the present invention, the Fc polypeptide is a fragment wherein 1, 2, 3, 4, 5, 6, 7, 8, 9, 10, 11, 12, 13, 14, 15, or 16 amino acids are removed from the N-terminus of SEQ ID NO: 12. In some embodiments, the Fc polypeptide includes mutations as set forth in Table B, relative to SEQ ID NO: 12.

TABLE B
Substitutions relative to SEQ ID NO: 12 for exemplary Fc Polypeptides

| Fc | 1 | 6 | 12 | 15 | 24 | 38 | 40 | 42 | 58 | 69 | 80 | 98 | 101 | 142 | 144 |
|---|---|---|---|---|---|---|---|---|---|---|---|---|---|---|---|
| M3 | A1V | | | | | | | | | | | | | D124E | L144M |
| M8 | A1V | | | | | | | | E58Q | E69Q | E80Q | D98N | N101D | D124E | L144M |
| M5 | A1Q | C6S | C12S | C15S | P24S | | | | | | | | | | |
| M4 | A1V | | | | | M38Y | | | | | | | | D142E | L144M |
| M4′ | A1V | | | | | | | T42E | | | | | | D142E | L144M |
| M5′ | A1V | | | | | M38Y | S40T | | | | | | | D142E | L144M |
| M6 | A1V | | | | | M38Y | S40T | T42E | | | | | | D142E | L144M |
| M6′ | A1Q | C6S | C12S | C15S | P24S | M38Y | | | | | | | | | |
| M7 | A1Q | C6S | C12S | C15S | P24S | M38Y | S40T | | | | | | | | |
| M8′ | A1Q | C6S | C12S | C15S | P24S | M38Y | S40T | T42E | | | | | | | |

In some embodiments of the present invention, the Fc polypeptide is joined to the BChE polypeptide via a linker. In some embodiments, the linker consists of about 3 to 20 amino acids. Examples of sequences of linkers that can be used in accordance with the presently-disclosed subject matter include SEQ ID NO: 13, SEQ ID NO: 14, SEQ ID NO: 15, SEQ ID NO: 16, SEQ ID NO: 17, and SEQ ID NO: 18.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The terms “polypeptide”, “protein”, and “peptide”, which are used interchangeably herein, refer to a polymer of the protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein”, “polypeptide”, and “peptide” are used interchangeably herein when referring to a gene product. Thus, exemplary polypeptides include gene products, naturally occurring proteins, homologs, orthologs, paralogs, fragments and other equivalents, variants, and analogs of the foregoing.

The term “variant” refers to an amino acid sequence that is different from the reference polypeptide by one or more amino acids, e.g., one or more amino acid substitutions. For example, a butyrylcholinesterase (BChE) polypeptide variant differs from wild-type BChE by one or more amino acid substitutions, i.e., mutations.

An “effective amount” of butyrylcholinesterase polypeptide variant or pharmaceutical composition to be used in accordance with the presently-disclosed subject matter is intended to mean a nontoxic but sufficient amount of the agent, such that the desired ghrelin hydrolase effect is produced. Thus, the exact amount of the enzyme or a particular agent that is required will vary from subject to subject, depending on the species, age, and general condition of the subject, the severity of the condition being treated, the particular carrier or adjuvant being used and its mode of administration, and the like. Similarly, the dosing regimen should also be adjusted to suit the individual to whom the composition is administered and will once again vary with age, weight, metabolism, etc. of the individual. Accordingly, the “effective amount” of any particular butyrylcholinesterase polypeptide variant, or pharmaceutical composition thereof, will vary based on the particular circumstances, and an appropriate effective amount may be determined in each case of application by one of ordinary skill in the art using only routine experimentation.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about.” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### EXAMPLES

### Example 1: BChE Mutants with Improved Catalytic Efficiency Against Ghrelin

A human BChE mutant known as CocH3, which is capable of converting ghrelin to desacyl-ghrelin as wild-type BChE, has been shown to have a desired anti-obesity effect.³⁹ Specifically, mice on a high-fat diet gained significantly less body weight when treated weekly with 1 mg/kg Fc-fused CocH3, denoted as CocH3-Fc(M3), compared to control mice, though their food intake was similar. 39 CocH3 or CocH3-Fc(M3) is the only known ghrelin-hydrolyzing enzyme whose in vivo effects on a ghrelin-related disorder (obesity) has been tested through administration of the purified protein, although other enzymes have also been tested through the gene transfer.^{21,40-42} For practical use of the ghrelin hydrolase strategy to treat ghrelin-related disorders, it would be useful to identify BChE mutants with improved catalytic efficiency against ghrelin, as compared to BChE and CocH3.

In order to design human BChE mutants with improved catalytic efficiency against ghrelin (i.e., converting ghrelin to desacyl-ghrelin), the present inventors studied the fundamental reaction pathway for BChE-catalyzed hydrolysis of ghrelin.⁴³ Generally speaking, for the purpose of rational design of a BChE mutant with improved catalytic activity for a specific substrate, it is desirable to know the detailed catalytic mechanism. The present inventors carried out a computational modeling and simulation study⁴³ using a hybrid quantum mechanical/molecular mechanical (QM/MM)-free energy (QM/MM-FE) approach which revealed the reaction pathway for BChE-catalyzed hydrolysis of ghrelin at the acylated Ser3 side chain. It has been demonstrated⁴³ that the same catalytic residues, including catalytic triad (S198, H438, and E325) and oxyanion hole (G116, G117, and A199), used in the BChE-catalyzed hydrolysis of cocaine are also used in the BChE-catalyzed hydrolysis of ghrelin. According to the computational data,⁴³ the chemical process of BChE-catalyzed ghrelin hydrolysis consists of a single-step acylation stage and a two-step deacylation stage, and that the acylation is rate-determining for the BChE-catalyzed ghrelin hydrolysis. In light of the mechanistic understanding, the present inventors have rationally designed and identified multiple human BChE mutants with improved catalytic efficiency against ghrelin compared to CocH3 (See Table C).

Structure and mechanism-based enzyme redesign (SMBER). The details of the general SMBER approach were previously described.⁴⁴ Briefly, the SMBER approach⁴⁴ is a virtual screening process, which is based on the efficient transition-state modeling (energy minimization) using a classical force field and a previously-described strategy.⁴⁵ The virtual screening started from the rate-determining transition-state structure for BChE-catalyzed hydrolysis of ghrelin.⁴³

Enzyme preparation. The new BChE mutants were prepared using the same procedure used in the accomplished studies to prepare BChE mutants against cocaine.⁴⁶⁻⁴⁸ Briefly, primers were designed to introduce point mutations to human BChE using in a pRc/CMV expression plasmid. Site-directed mutagenesis was performed using the QuickChange method,⁴⁹ in which a pair of complementary mutagenic primers in a thermocycling reaction amplify the plasmid with pfu DNA polymerase. Each mutant was expressed in Chinese hamster ovary (CHO) cells in free-style CHO expression medium. Cells was first grown to a density of ˜1.0×10⁶ cells/ml in 2 L shake flask and transfected using TransIT-PRO Transfectio Kit. Cells were incubated at 37° C. in a CO₂ incubator for 5 days. The culture medium was then be harvested and purified by using a two-step method previously-described.^{47, 50}

Enzyme activity assay. The purified BChE mutants were evaluated for their catalytic activity against ghrelin by using a sensitive radiometric assay⁵¹ with [³H]-ghrelin labeled on its octanoic acid moiety of the acylated Ser3. The experimental material, [³H]-ghrelin (˜80 Ci/mmol), was obtained through custom synthesis by Quotient Bioresearch (Cardiff. UK). The assay is based on toluene extraction of ghrelin hydrolysis product [³H]-octanoic acid, which is similar to the previously-described radiometric assay for BChE-catalyzed hydrolysis of (−)-cocaine with [³H]-(−)-cocaine based on the toluene extraction of (−)-cocaine hydrolysis product [³H]-benzoic acid.^{44-45,47-48,52} The toluene extraction of [³H]-octanoic acid is an effective solvent-partitioning procedure with aqueous and toluene phases. At a very low pH, [³H]-octanoic acid is protonated and the protonated [³H]-octanoic acid exists in toluene phase, while [³H]-ghrelin exists in aqueous phase. To determine the Michaelis-Menten kinetics, reactions took place at 37° C. and pH 7.4 with variable concentrations of ghrelin. Reactions were stopped with 0.1 N HCl. [³H]-octanoic acid was extracted to the organic (toluene) phase by 1 ml of toluene and vortexing. The concentrations of [³H]-octanoic acid in toluene phase and un-reacted [³H]-ghrelin in aqueous phase were determined through scintillation counting.

TABLE C
Relative catalytic efficiency against ghrelin determined
by the in vitro enzyme activity assay.

| | Relative catalytic |
|---|---|
| | efficiency for ghrelin |
| Enzyme | (Relative to CocH3) |
| CocH3^{a} | 1 |
| Wild-type human BChE (SEQ ID NO: 2) | 1.5 |
| F398I (SEQ ID NO: 3) | 2 |
| T284S/P285I/F398I (SEQ ID NO: 4) | 3 |
| T284S/P285I/V288I (SEQ ID NO: 5) | 3 |
| T284S/P285I/V288I/F398I (SEQ ID NO: 6) | 3 |
| A199S/S287G/A328W/Y332G (SEQ ID NO: 7) | 2 |
| A199S/F227A/P285Q/S287G/A328W/Y332G | 3 |
| (SEQ ID NO: 8) | |
| A199S/F227A/P285S/S287G/A328W/Y332G | 2 |
| (SEQ ID NO: 9) | |
| A199S/F227A/P285A/S287G/A328W/Y332G | 3 |
| (SEQ ID NO: 10) | |

^{a}Previously tested BChE mutant (known as CocH3—A199S/F227A/S287G/A328W/Y332G (SEQ ID NO: 11)) in mice for its anti-obesity effects (ref.³⁹).

All the BChE mutants listed in Table C are contemplated for treatment of a variety of ghrelin-related disorders, including but not limited to obesity, hyperphagia and Prader-Willi syndrome, hyperglycemia, sleep disorder, emotional disorder, T2D, cancer, and substance use disorders. The substance use disorders include but not limited to mental and behavioral disorders due to single or multiple substance use, for example, methamphetamines, opioids (e.g. heroin, morphine, oxycodone, hydrocodone, fentanyl and its derivatives etc.), cocaine, alcohol, and nicotine.

### Example 2: Ghrelin Deacylase for Treatment of Substance Use Disorders (SUDs)

Current therapeutic development efforts for treatment of substance use disorders (SUDs) have been focused mainly on small-molecule compounds that can bind with specific receptors/transporters (targets) in the brain to attenuate the physiological, subjective, and/or reinforcing effects of the drugs, although there are also efforts to develop biologics including drug-binding monoclonal antibodies or vaccines that produce the desirable antibodies that can bind tightly to the drugs so as to prevent crossing the blood-brain barrier (BBB). One of the promising targets is ghrelin or growth hormone (GH) secretagogue receptor (GHSR) or GHS-R1A.²⁷⁻³² In ghrelin receptor knockout mice and mice treated with JMV2959 (an antagonist specific for GHSR),³³ alcohol did not increase locomotion, induce the release of DA into the NAc, or establish a conditioned place preference (CPP).²⁷ Studies on nicotine and psychostimulants demonstrated that GHSR antagonism with JMV2959 attenuated nicotine-, amphetamine-, and cocaine-induced hyperactivity, NAc DA release, and CPP.²⁸⁻²⁹ Treatment with JMV2959 reduced morphine-induced hyperactivity and decreased NAc DA levels in mice.³⁰ Administration of JMV2959 significantly and dose-dependently attenuated morphine-induced CPP and DA sensitization,³¹ and attenuated methamphetamine (METH) self-administration, tendency to relapse, and METH-induced CPP.³² JMV2959 also significantly reduced the fentanyl-seeking/relapse-like behavior in rats.³⁴ GHSR antagonism is one of the NIDA's 10 Most-Wanted targets³⁵ for opioid user disorder (OUD) medication development. However, due to the GHSR's diverse regulatory roles associated with its constitutive activity, GHSR antagonism could also result in adverse effects.³⁸ Thus, alternative strategies targeting ghrelin itself are desired.

It is contemplated that it would be beneficial to downregulate AG and upregulate DAG or, in overall, downregulate the AG/DAG ratio. However, recently published experimental studies have indicated that downregulating circulating ghrelin would not work for treatment of SUDs. For example, Spiegelmer NOX-B11-2 (a synthetic l-oligonucleotide), which was expected to stoichiometrically bind circulating ghrelin and prevent it from crossing the BBB.⁵⁴ was used in a study on alcohol reward.⁵⁵ An intraperitoneal (IP) injection of 20 mg/kg NOX-B11-2 was administered 60 minutes prior to alcohol (1.75 g/kg, IP) exposure, and it did not attenuate alcohol-induced locomotion, NAc DA release, or CPP in mice, nor did it alter alcohol intake in rats.⁵⁵ NOX-B11-2 reduced food intake 1 hour after administration, but no change in food intake was observed at the 4 and 24 hour time points.⁵⁵ Body weight was not altered.⁵⁵ For another example, a ghrelin vaccine (150 μL/vaccine, delivering a dose of 50 μg of the ghrelin hapten conjugate) was administered in mice.⁵⁶ Throughout this study, the body weight of the mice decreased, but cocaine-induced CPP establishment and hyperactivity were not attenuated.⁵⁶

Disclosed herein is the use of an efficient ghrelin deacylase, which can effectively convert AG to DAG and, thus, downregulate the AG/DAG ratio. Such a ghrelin deacylase could be useful as a safe and effective ghrelin modulator to attenuate substance rewarding effects and treat SUDs (dependence or addiction), including methamphetamine (METH) and opioid use disorders etc. An efficient exogenous ghrelin deacylase is expected to be as effective as a selective GHSR antagonist in attenuating the reward effects, but without affecting the naturally high constitutive activity³⁶ of GHSR.

BChE-M02 is an exemplary ghrelin deacylases as disclosed herein, which is the A199S/F227A/P285A/S287G/A328W/Y332G (SEQ ID NO: 10) mutant of human BChE with an improved catalytic activity compared to wild-type BChE for ghrelin deacylation. As disclosed herein, BChE-M02 is capable of converting AG to DAG and, thus, attenuating substance (e.g. an opioid or METH)-induced hyperactivity and the substance rewarding effects.

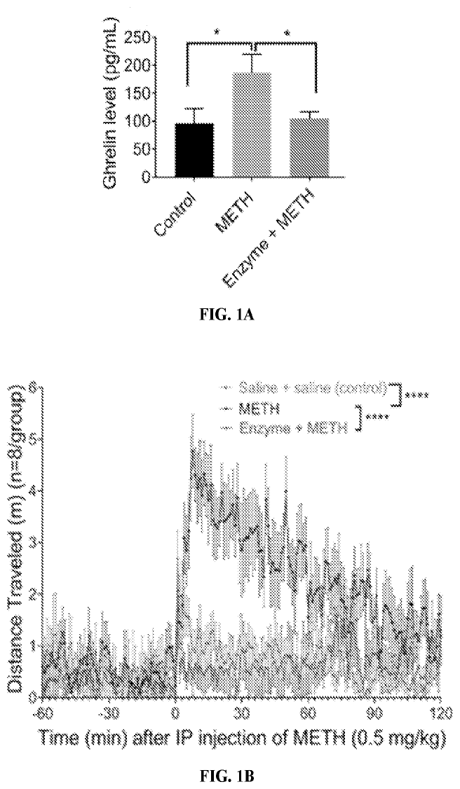

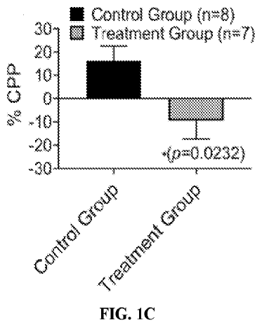

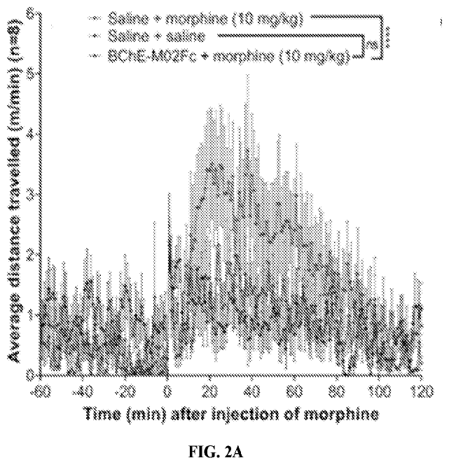

For example, administration of a dose (26 mg/kg) of BChE-M02 significantly reduced the blood AG level (FIG. 1A) such that the AG/DAG ratio changed from 0.41 to 0.23, effectively blocked METH-induced hyperactivity in mice (FIG. 1B), and effectively blocked METH-induced CPP (FIG. 1C) in mice, without any toxicity sign noted after the BChE-M02 administration.

In addition, IV administration of BChE-M02 did not significantly affect the baseline locomotor activity and food self-administration in rats (data not shown), which is consistent with results recently reported for another BChE mutant.⁵⁷ The IP dose of METH (METH hydrochloride, 0.5 mg/kg) used in the studies to obtain the data in FIG. 1A-1C was the same as the previously used IP dose of 2.69 μmol/kg METH for METH-induced hyperactivity studies. 58 Accordingly, the ghrelin deacylase and the GHSR antagonist JMV2959 indeed have the similar effects in terms of attenuating METH-induced hyperactivity. These data consistently support administration of ghrelin deacylase as a therapeutic approach to treatment of SUDs.

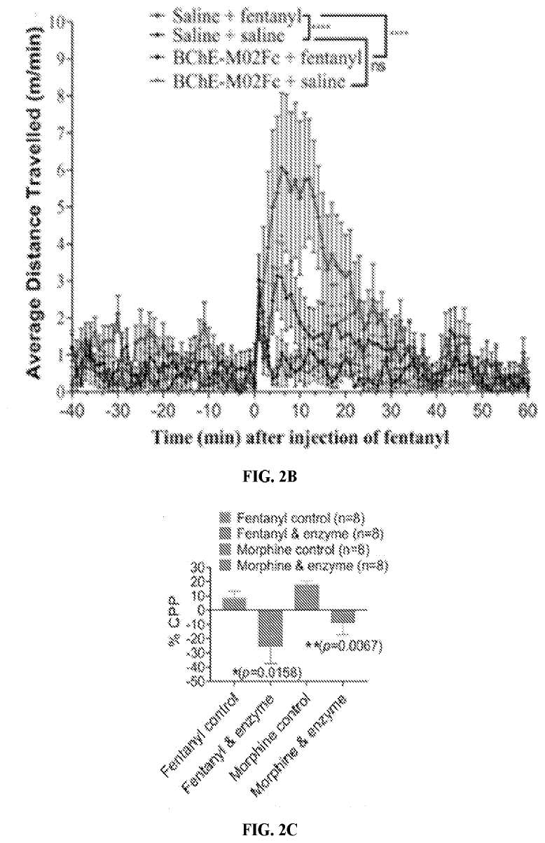

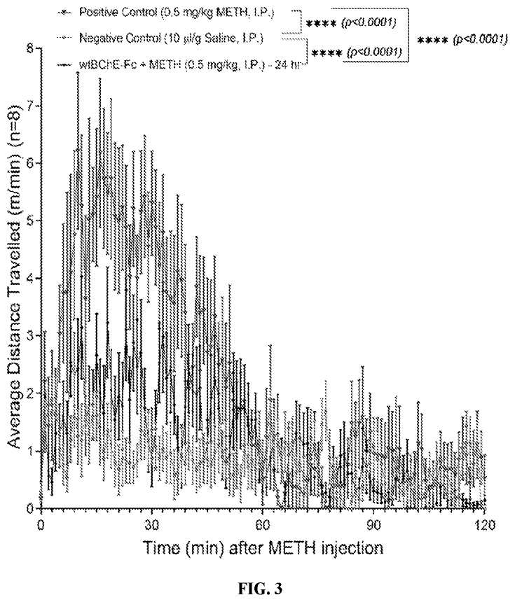

Further, with consideration to the previous development of a long-acting cocaine hydrolase (CocH-Fc) for cocaine abuse treatment,^{52,57,59,60} a long-acting Fc-fusion protein form of BChE-M02, denoted as BChE-M02Fc, has also been developed as disclosed herein. As seen in FIG. 2A-2C, BChE-M02Fc at a dose of 15 mg/kg (IV) significantly attenuated morphine- and fentanyl-induced hyperactivity and CPP in mice. Additionally, with reference to FIG. 3, the effect of recombinant wild-type BChE fused with Fc of human IgG1 (denoted as wtBChE-Fc) was tested on METH-induced hyperactivity in mice (n=8 per group). Mice were given wtBChE-Fc at a dose of 26 mg/kg (IV) 2 h before the METH administration.

### Example 3: Ghrelin Deacylase for Treatment of Hyperglycemia and Diabetes

Studies were also conducted to explore the impact of treatment on hyperglycemia and diabetes.

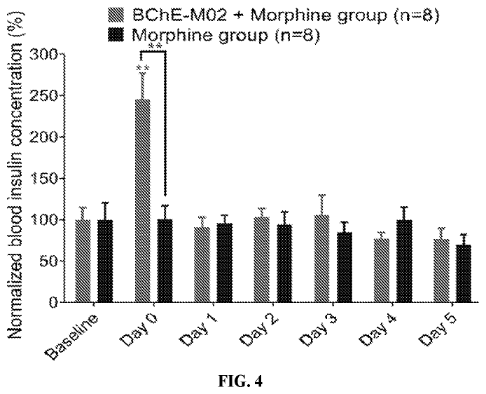

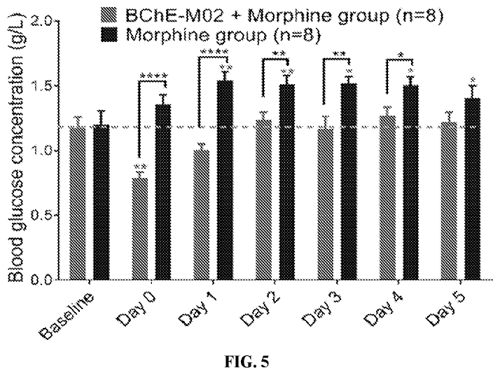

With reference to FIG. 4, the insulin level in morphine-challenged mice was assessed. A single 15 mg/kg dose of BChE-M02Fc (IV) was administered at 3 h before IP injection of 10 mg/kg morphine on Day 0, followed by daily 10 mg/kg morphine (IP) challenge on Days 1 to 5. Blood samples were collected at 2 h after the morphine administration. These data demonstrate that administration of BChE-M02 can significantly increase the blood insulin level.

With reference to FIG. 5, the glucose level in morphine-challenged mice was assessed. A single 15 mg/kg dose of BChE-M02Fc (IV) was injected at 3 h before IP injection of 10 mg/kg morphine on Day 0, followed by daily 10 mg/kg morphine (IP) challenge on Days 1 to 5. Blood samples were collected at 2 h after the morphine administration. These data demonstrate that administration of BChE-M02 can significantly decrease the blood glucose level.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Kojima, M.; Hosoda, H.; Date, Y.; Nakazato, M.; Matsuo, H.; Kangawa, K., Ghrelin is a growth-hormone-releasing acylated peptide from stomach. Nature 1999, 402(6762), 656-60.
2. Gutierrez, J. A.; Solenberg, P. J.; Perkins, D. R.; Willency, J. A.; Knierman, M. D.; Jin, Z.; Witcher, D. R.; Luo, S.; Onyia, J. E.; Hale, J. E., Ghrelin octanoylation mediated by an orphan lipid transferase. Proceedings of the National Academy of Sciences of the United States of America 2008, 105 (17), 6320-6325.
3. Yang, J.; Brown, M. S.; Liang, G.; Grishin, N. V.; Goldstein, J. L., Identification of the Acyltransferase that Octanoylates Ghrelin, an Appetite-Stimulating Peptide Hormone. Cell 2008, 132(3), 387-396.
4. Cleverdon, E. R.; McGovern-Gooch, K. R.; Hougland, J. L., The octanoylated energy regulating hormone ghrelin: An expanded view of ghrelin's biological interactions and avenues for controlling ghrelin signaling. Molecular Membrane Biology 2016, 33(6-8), 111-124.
5. Jenny, T.; Nimita, D.; Ganesh, M. M.; Harold, W. D.; Bruce, D. G.; Michael, O. T.; Matthias, H. T.; David, D. A.; Pankaj, B. D., The pharmacokinetics of acyl, des-acyl, and total ghrelin in healthy human subjects. European Journal of Endocrinology 2013, 168(6), 821-828.
6. May, B., Synthesis, secretion, and deacylation of Ghrelin (reactome.org/content/detail/R-HSA-422085). Reactome 2009.
7. Satou, M.; Sugimoto, H., The Study of Ghrelin Deacylation Enzymes. Methods in enzymology 2012, 514, 165-179.
8. Kyle, T.; Hignett, W., Ghrelin, the “Go” Hormone (www.obesityaction.org/wp-content/uploads/Ghrelin-the-GO-Hormone.pdf) (Last seen: Sep. 11, 2020).
9. Sakao, Y.; Ohashi, N.; Sugimoto, M.; Ichikawa, H.; Sahara, S.; Tsuji, T.; Kato, A.; Fujigaki, Y.; Sugimoto, K.; Furuta, T.; Yasuda. H., Gender Differences in Plasma Ghrelin Levels in Hemodialysis Patients. Therapeutic Apheresis and Dialysis 2019, 23 (1), 65-72.
10. Tong, J.; Dave, N.; Mugundu, G. M.; Davis, H. W.; Gaylinn, B. D.; Thorner, M. O.; Tschop, M. H.; D'Alessio, D.; Desai, P. B., The Pharmacokinetics of Acyl, Desacyl and Total Ghrelin in Healthy Human Subjects. Eur. J. Endocrinol. 2013, 168, 821-828.
11. Dornonville de la Cour, C.; Lindqvist, A.; Egecioglu. E.; Tung, Y. C. L.; Surve, V.; Ohlsson, C.; Jansson, J. O.; Erlanson-Albertsson, C.; Dickson, S. L.; Håkanson, R., Ghrelin treatment reverses the reduction in weight gain and body fat in gastrectomised mice. Gut 2005, 54 (7), 907-913.
12. Handjieva-Darlenska, T.; Boyadjieva. N.; Takov, K., Emerging Pharmacotherapies to Fight Obesity and Related Disorders. J Obes Weight Loss Ther 2014, 4(226), 2.
13. Shearman, L. P.; Wang, S.-P.; Helmling, S.; Stribling, D. S.; Mazur, P.; Ge, L.; Wang, L.; Klussmann, S.; Macintyre, D. E.; Howard, A. D.; Strack, A. M., Ghrelin Neutralization by a Ribonucleic Acid-SPM Ameliorates Obesity in Diet-Induced Obese Mice. Endocrinology 2006, 147(3), 1517-1526.
14. Zorrilla, E. P.; Iwasaki, S.; Moss, J. A.; Chang, J.; Otsuji, J.; Inoue, K.; Meijler, M. M.; Janda. K. D., Vaccination against weight gain. Proceedings of the National Academy of Sciences 2006, 103 (35), 13226-13231.
15. Yang, J.; Zhao, T.-J.; Goldstein. J. L.; Brown, M. S., Inhibition of ghrelin O-acyltransferase (GOAT) by octanoylated pentapeptides. Proceedings of the National Academy of Sciences 2008, 105 (31), 10750-10755.
16. Schellekens, H.; Dinan, T. G.; Cryan, J. F., Lean mean fat reducing “ghrelin” machine: Hypothalamic ghrelin and ghrelin receptors as therapeutic targets in obesity. Neuropharmacology 2010, 58 (1), 2-16.
17. Barnett, B. P.; Hwang. Y.; Taylor, M. S.; Kirchner, H.; Pfluger, P. T.; Bernard, V.; Lin, Y.-y.; Bowers, E. M.; Mukherjee, C.; Song, W.-J.; Longo, P. A.; Leahy, D. J.; Hussain, M. A.; Tschöp, M. H.; Boeke, J. D.; Cole, P. A., Glucose and Weight Control in Mice with a Designed Ghrelin O-Acyltransferase Inhibitor. Science 2010, 330 (6011), 1689-1692.
18. DelParigi, A.; Tschöp, M.; Heiman, M. L.; Salbe, A. D.; Vozarova, B.; Sell, S. M.; Bunt, J. C.; Tataranni. P. A., High Circulating Ghrelin: A Potential Cause for Hyperphagia and Obesity in Prader-Willi Syndrome. The Journal of Clinical Endocrinology & Metabolism 2002, 87, 5461-5464. doi.org/10.1210/jc.2002-020871.
19. Mani, B. K.; Shankar, K.; Zigman, J. M., Ghrelin's Relationship to Blood Glucose. Endocrinology 2019, 160, 1247-1261. doi.org/10.1210/en.2019-00074.
20. Taheri, S.; Lin, L.; Austin, D.; Young, T.; Mignot, E., Short Sleep Duration Is Associated with Reduced Leptin, Elevated Ghrelin, and Increased Body Mass Index. PLOS Med. 2004, 1(3), e62. doi: 10.1371/journal.pmed.0010062.
21. Brimijoin, S.; Gao, Y.; Geng, L.; Chen, V. P., Treating Cocaine Addiction, Obesity, and Emotional Disorders by Viral Gene Transfer of Butyrylcholinesterase. Front. Pharmacol. 2018, 9, 112. doi: 10.3389/fphar.2018.00112. eCollection 2018.
22. Poher, A.-L.; Tschöp, M. H.; Müller, T. D., Ghrelin regulation of glucose metabolism. Peptides 2018, 100, 236-242.
23. Lin, T.-C.; Hsiao, M., Ghrelin and cancer progression. Biochim. Biophys. Acta Rev. Cancer 2017, 1868(1), 51-57. doi: 10.1016/j.bbcan.2017.02.002.
24. Koob, G. F., 10 percent of US adults have drug use disorder at some point in their lives-75 percent report not receiving any form of treatment. NIH-Turning Discovery Into Health (www.nih.gov/news-events/news-releases/10-percent-us-adults-have-drug-use-disorder-some-point-their-lives). Last seen: Sep. 11, 2020.
25. Fiellin, D. A.; Schottenfeld, R. S.; Cutter, C. J.; Moore, B. A.; Barry, D. T.; O'Connor, P. G., Primary care-based buprenorphine taper vs maintenance therapy for prescription opioid dependence: a randomized clinical trial. JAMA Intern. Med. 2014, 174 1947-1954.
26. Smyth, B. P.; Barry, J.; Keenan. E.; Ducray, K., Lapse and relapse following inpatient treatment of opiate dependence. Ir. Med. J. 2010, 103 176-179.
27. Jerlhag, E.; Egecioglu, E.; Landgren, S.; Salome, N.; Heilig, M.; Moechars, D.; Datta, R.; Perrissoud, D.; Dickson, S. L.; Engel, J. A., Requirement of central ghrelin signaling for alcohol reward. Proc Natl Acad Sci USA 2009, 106(27), 11318-23.
28. Jerlhag, E.; Egecioglu, E.; Dickson, S. L.; Engel, J. A., Ghrelin receptor antagonism attenuates cocaine- and amphetamine-induced locomotor stimulation, accumbal dopamine release, and conditioned place preference. Psychopharmacology (Berl) 2010, 211(4), 415-22.
29. Jerlhag, E.; Engel, J. A . . . . Ghrelin receptor antagonism attenuates nicotine-induced locomotor stimulation, accumbal dopamine release and conditioned place preference in mice. Drug Alcohol Depend 2011, 117(2-3), 126-31.
30. Engel, J. A.; Nylander, I.; Jerlhag. E., A ghrelin receptor (GHS-R1A) antagonist attenuates the rewarding properties of morphine and increases opioid peptide levels in reward areas in mice. European neuropsychopharmacology: the journal of the European College of Neuropsychopharmacology 2015, 25(12), 2364-71.
31. Jerabek, P.; Havlickova. T.; Puskina, N.; Charalambous, C.; Lapka, M.; Kacer, P.; Sustkova-Fiserova, M., Ghrelin receptor antagonism of morphine-induced conditioned place preference and behavioral and accumbens dopaminergic sensitization in rats. Neurochem Int 2017, 110, 101-113.
32. Havlickova, T.; Charalambous, C.; Lapka, M.; Puskina, N.; Jerabek, P.; Sustkova-Fiserova, M., Ghrelin Receptor Antagonism of Methamphetamine-Induced Conditioned Place Preference and Intravenous Self-Administration in Rats. Int. J. Mol. Sci. 2018, 19(10), E2925. doi: 10.3390/ijms19102925.
33. Moulin, A.; Demange, L.; Berge, G.; Gagne, D.; Ryan, J.; Mousseaux, D.; Heitz, A.; Perrissoud, D.; Locatelli, V.; Torsello, A.; Galleyrand, J. C.; Fehrentz, J. A.; Martinez, J., Toward potent ghrelin receptor ligands based on trisubstituted 1,2,4-triazole structure. 2. Synthesis and pharmacological in vitro and in vivo evaluations. Journal of medicinal chemistry 2007, 50 (23), 5790-806.
34. Sustkova-Fiserova, M.; Puskina, N.; Havlickova, T.; Lapka, M.; Syslova, K.; Pohorala, V.; Charalambous, C., Ghrelin receptor antagonism of fentanyl-induced conditioned place preference, intravenous self-administration, and dopamine release in the nucleus accumbens in rats. Addict. Biol. 2019 Nov. 6: e12845. doi: 10.1111/adb.12845.
35. Rasmussen, K.; White, D. A.; Acri, J. B., NIDA's medication development priorities in response to the Opioid Crisis: ten most wanted. Neuropsychopharmacology 2019, 44, 657-659.
36. Els, S.; Beck-Sickinger, A. G.; Chollet, C., Ghrelin receptor: high constitutive activity and methods for developing inverse agonists. Methods Enzymol. 2010, 485, 103-121. doi: 10.1016/B978-0-12-381296-4.00006-3.
37. Dickson. S. L.; Egecioglu, E.; Landgren, S.; Skibicka, K. P.; Engel, J. A.; Jerlhag, E., The role of the central ghrelin system in reward from food and chemical drugs. Mol. Cell Endocrinol. 2011, 340(1), 80-87.
38. M'Kadmi, C.; Leyris, J.-P.; Onfroy, L.; Gales, C.; Saulière, A.; Gagne, D.; Damian, M.; Mary, S.; Maingot, M.; Denoyelle, S.; Verdie, P.; Fehrentz, J.-A.; Martinez, J.; Banères, J.-L.; Marie. J., Agonism, Antagonism, and Inverse Agonism Bias at the Ghrelin Receptor Signaling. J. Biol. Chem. 2015, 290(45), 27021-27039. doi: 10.1074/jbc.M115.659250.
39. Zheng. X.; Zhang, T.; Zheng, F.; Zhan, C.-G., Potential anti-obesity effects of a long-acting cocaine hydrolase. Chem. Biol. Interact. 2016, 259, 99-103.
40. Chen, V. P.; Gao, Y.; Geng. L.; Brimijoin, S., Butyrylcholinesterase gene transfer in obese mice prevents postdieting body weight rebound by suppressing ghrelin signaling. Proc. Natl. Acad. Sci. USA 2017, 114, 10960-10965.
41. Chen, V. P.; Gao, Y.; Geng, L.; Stout, M. B.; Jensen, M. D.; Brimijoin, S., Butyrylcholinesterase Deficiency Promotes Adipose Tissue Growth and Hepatic Lipid Accumulation in Male Mice on High-Fat Diet. Endocrinology 2016, 157, 3086-3095.
42. Chen, V. P.; Gao, Y.; Geng, L.; Parks, R. J.; Pang, Y. P.; Brimijoin, S., Plasma butyrylcholinesterase regulates ghrelin to control aggression. Proc. Natl. Acad. Sci. USA 2015, 112, 2251-2256.
43. Yao, J.; Yuan, Y.; Zheng, F.; Zhan, C.-G., Unexpected reaction pathway for butyrylcholinesterase-catalyzed inactivation of ‘hunger hormone’ ghrelin. Sci. Rep. 2016, 6, 22322: doi: 10.1038/srep22322.
44. Zheng. F.; Xue, L.; Hou, S.; Liu, J.; Zhan, M.; Yang, W.; Zhan, C.-G., A highly efficient cocaine-detoxifying enzyme obtained by computational design. Nature Commun. 2014, 5, 3457. doi: 10.1388/ncomms4457.
45. Pan, Y.; Gao, D.; Yang, W.; Cho, H.; Yang, G.; Tai, H.-H.; Zhan, C.-G., Computational redesign of human butyrylcholinesterase for anticocaine medication. Proc. Natl. Acad. Sci. USA 2005, 102 (46), 16656-16661.
46. Zheng, F.; Xue, L.; Hou, S.; Liu, J.; Zhan, M.; Yang, W.; Zhan, C. G., A highly efficient cocaine-detoxifying enzyme obtained by computational design. Nat Commun 2014, 5, 3457.
47. Xue, L.; Ko. M.-C.; Tong, M.; Yang, W.; Hou, S.; Fang, L.; Liu, J.; Zheng, F.; Woods, J. H.; Tai. H.-H.; Zhan, C.-G., Design, preparation, and characterization of high-activity mutants of human butyrylcholinesterase specific for detoxification of cocaine. Mol. Pharmacol. 2011, 79, 290-297.
48. Zheng. F.; Yang, W. C.; Ko, M. C.; Liu, J. J.; Cho, H.; Gao, D. Q.; Tong, M.; Tai, H. H.; Woods, J. H.; Zhan, C.-G., Most Efficient Cocaine Hydrolase Designed by Virtual Screening of Transition States. J. Am. Chem. Soc. 2008, 130 (36), 12148-12155.
49. Braman, J.; Papworth, C.; Greener, A., Site-directed mutagenesis using double-stranded plasmid DNA templates. Methods Mol Biol 1996, 57, 31-44.
50. Xue, L.; Hou, S.; Tong, M.; Fang. L.; Chen, X.; Jin, Z.; Tai, H.-H.; Zheng. F.; Zhan. C.-G., Preparation and in vivo characterization of a cocaine hydrolase engineered from human butyrylcholinesterase for metabolizing cocaine. Biochem. J. 2013, 453, 447-454.
51. Chen, V. P.; Gao, Y.; Geng. L.; Brimijoin, S., Radiometric assay of ghrelin hydrolase activity and 3H-ghrelin distribution into mouse tissues. Biochem Pharmacol 2015, 98(4), 732-739.
52. Chen, X.; Xue, L.; Hou, S.; Jin, Z.; Zhang, T.; Zheng, F.; Zhan, C.-G., Long-acting cocaine hydrolase for addiction therapy. Proc. Natl. Acad. Sci. USA 2016, 113, 422-427.
53. Chen, V. P.; Gao, Y.; Geng. L.; Brimijoin, S., Butyrylcholinesterase regulates central ghrelin signaling and has an impact on food intake and glucose homeostasis. Int J Obes 2017, 41(9), 1413-1419.
54. Helmling, S.; Maasch. C.; Eulberg, D.; Buchner, K.; Schröder, W.; Lange, C.; Vonhoff, S.; Wlotzka. B.; Tschöp, M. H.; Rosewicz, S.; Klussmann, S., Inhibition of ghrelin action in vitro and in vivo by an RNA-Spiegelmer. Proceedings of the National Academy of Sciences of the United States of America 2004, 101 (36), 13174-13179.
55. Jerlhag, E.; Ivanoff, L.; Vater. A.; Engel, J. A., Peripherally circulating ghrelin does not mediate alcohol-induced reward and alcohol intake in rodents. Alcohol Clin Exp Res 2014, 38 (4), 959-68.
56. Wenthur, C. J.; Gautam. R.; Zhou, B.; Vendruscolo, L. F.; Leggio, L.; Janda, K. D., Ghrelin Receptor Influence on Cocaine Reward is Not Directly Dependent on Peripheral Acyl-Ghrelin. Sci Rep 2019, 9(1), 1841.
57. Zhang, T.; Wei, H.; Deng. J.; Zheng, F.; Zhan, C.-G., Clinical potential of a rationally engineered enzyme for treatment of cocaine dependence: Long-lasting blocking of the psychostimulant, discriminative stimulus, and reinforcing effects of cocaine. Neuropharmacology 2020, 176, 108251. doi: 10.1016/j.neuropharm.2020.108251.
58. Tapia, M. A.; Lever, J. R.; Lever, S. Z.; Will, M. J.; Park, E. S.; Miller, D. K., Sigma-1 receptor ligand PD144418 and sigma-2 receptor ligand YUN-252 attenuate the stimulant effects of methamphetamine in mice. Psychopharmacology 2019, 236(11), 3147-3158. doi: 10.1007/s00213-019-05268-2.
59. Chen, X.; Cui, W.; Deng, J.; Hou, S.; Zhang, J.; Ding, X.; Zheng, X.; Wei, H.; Zhou, Z.; Kim, K.; Zhan, C.-G.; Zheng, F., Development of Fc-fused Cocaine Hydrolase for Cocaine Addiction: Catalytic and Pharmacokinetic Properties. AAPS J 2018, 20, 53. doi: 10.1208/s12248-018-0214-9.
60. Zheng, F.; Chen, X.; Kim, K.; Zhang, T.; Huang, H.; Zhou, S.; Zhang, J.; Jin, Z.; Zhan, C.-G., Structure-Based Design and Discovery of a Long-Acting Cocaine Hydrolase Mutant with Improved Binding Affinity to Neonatal Fc Receptor for Treatment of Cocaine Abuse. AAPS J. 2020, 22, 62. doi: 10.1208/s12248-020-00442-3.
61. U.S. Pat. Nos. 7,438,904, 7,731,957, 7,919,082, 8,193,327, 8,399,644, 8,835,150, 9,206,403, 7,740,840, 7,892,537, 8,206,703, 8,846,887, 9,175,274, 9,415,092, 8,592,193, 8,945,901, and 9,365,841, and U.S. patent application Ser. No. 17/129,736

It will be understood that various details of the presently disclosed subject matter can be changed without departing from the scope of the subject matter disclosed herein. Furthermore, the foregoing description is for the purpose of illustration only, and not for the purpose of limitation.

1. A method of inactivating ghrelin, comprising administering a ghrelin hydrolase to convert the ghrelin to desacyl-ghrelin, wherein the ghrelin hydrolase comprises a butyrylcholinesterase (BChE) polypeptide variant comprising an amino acid sequence having 1, 2, 3, 4, 5, or 6 mutations relative to the amino acid sequence of SEQ ID NO: 2, said mutations selected from the group consisting of A199S, F227A, T284S, P285I or P285A or P285Q or P285S, S287G, V288I, A328W, Y332G, and F398I, and wherein the amino acid sequence of the BChE polypeptide variant is optionally truncated relative to the amino acid sequence of SEQ ID NO: 2, such that one or more residues from 1 to 67 and/or one or more residues from 443 to 574 is truncated.

2. The method of claim 1, wherein the a BChE polypeptide variant comprises an amino acid sequence having 1, 2, 3, or 4 mutations relative to the amino acid sequence of SEQ ID NO: 2, said mutations selected from the group consisting of T284S, P285I, V288I, F398I; and wherein the amino acid sequence of the BChE polypeptide variant is optionally truncated relative to the amino acid sequence of SEQ ID NO: 2, such that one or more residues from 1 to 67 and/or one or more residues from 443 to 574 is truncated.

3. The method of claim 1, wherein the BChE polypeptide variant comprising an amino acid sequence having 1, 2, 3, 4, 5, or 6 mutations relative to the amino acid sequence of SEQ ID NO: 2, said mutations selected from the group consisting of A199S, F227A, P285A or P285Q or P285S, S287G, A328W, and Y332G; and wherein the amino acid sequence of the BChE polypeptide variant is optionally truncated relative to the amino acid sequence of SEQ ID NO: 2, such that one or more residues from 1 to 67 and/or one or more residues from 443 to 574 is truncated.

4. The method of claim 1, wherein the BChE polypeptide variant comprises an amino acid sequence wherein residues 530 to 574 are truncated, such that the amino acid sequence has 529 amino acids.

5. The method of claim 1, wherein the BChE polypeptide variant comprises the amino acid sequence of any one of SEQ ID NOS: 3-10.

6. The method of claim 1, and further comprising, joined to an N- or C-terminal end of the BChE polypeptide variant, a Fc polypeptide has the sequence of SEQ ID NO: 12, or a fragment thereof, wherein the Fc polypeptide or fragment thereof includes 0 to 8 amino acid substitutions at 0 to 8 of residues selected from 1, 6, 12, 15, 24, 38, 40, 42, 58, 69, 80, 98, 101, 142, and 144.

7. The method of claim 6, wherein the Fc polypeptide is joined to the BChE polypeptide via a linker.

8. The method of claim 1, and further comprising downregulating circulating ghrelin in the plasma of a subject by administering the ghrelin hydrolase to the subject.

9. The method of claim 8, wherein the subject is in need of treatment for a condition selected from the group consisting of: obesity, hyperphagia and Prader-Willi syndrome, hyperglycemia, sleep disorder, emotional disorder, type 2 diabetes (T2D), cancer, and substance use disorders.

10. The method of claim 8, wherein the subject is in need of treatment for polysubstance use disorder involving two or more substances selected from the group consisting of: methamphetamine, cocaine, alcohol, nicotine, and opioids.

12. The method of claim 1, wherein the ghrelin hydrolase is provided in a composition together with a pharmaceutically-acceptable carrier.

13. A butyrylcholinesterase (BChE) polypeptide variant comprising an amino acid sequence having 1, 2, 3, or 4 mutations relative to the amino acid sequence of SEQ ID NO: 2, said mutations selected from the group consisting of T284S, P285I, V288I, F398I; and wherein the amino acid sequence of the BChE polypeptide variant is optionally truncated relative to the amino acid sequence of SEQ ID NO: 2, such that one or more residues from 1 to 67 and/or one or more residues from 443 to 574 is truncated.

14. The BChE polypeptide variant of claim 13, wherein residues 530 to 574 are truncated, such that the amino acid sequence has 529 amino acids.

15. The BChE polypeptide variant of claim 13, comprising the amino acid sequence of any one of SEQ ID NOS: 3-6.

16. The BChE polypeptide variant of claim 13, and further comprising, joined to an N- or C-terminal end of the BChE polypeptide variant, a Fc polypeptide has the sequence of SEQ ID NO: 12, or a fragment thereof, wherein the Fc polypeptide or fragment thereof includes 0 to 8 amino acid substitutions at 0 to 8 of residues selected from 1, 6, 12, 15, 24, 38, 40, 42, 58, 69, 80, 98, 101, 142, and 144.

17. The BChE polypeptide variant of claim 16, wherein the Fc polypeptide is joined to the BChE polypeptide via a linker.

18. The BChE polypeptide variant of claim 13, provided in a composition together with a pharmaceutically-acceptable carrier.

19. A method of inactivating ghrelin in a subject in need of treatment for a substance use disorder, comprising administering to the subject a composition comprising a butyrylcholinesterase (BChE) or BChE polypeptide variant, and a pharmaceutically acceptable carrier, wherein the BChE polypeptide variant comprises an amino acid sequence having 1, 2, 3, 4, 5, or 6 mutations relative to the amino acid sequence of SEQ ID NO: 2, said mutations selected from the group consisting of A199S, F227A, T284S, P285I or P285A or P285Q or P285S, S287G, V288I, A328W, Y332G, and F398I, and wherein the amino acid sequence of the BChE polypeptide variant is optionally truncated relative to the amino acid sequence of SEQ ID NO: 2, such that one or more residues from 1 to 67 and/or one or more residues from 443 to 574 is truncated.

20. The method of claim 19, wherein the subject is in need of treatment for polysubstance use disorder involving two or more substances selected from the group consisting of: methamphetamine, cocaine, alcohol, nicotine, and opioids.

21-30. (canceled)
